SEQUENCE LISTING (85 sequences)

SEQ ID NO: 1 - protein, 139 aa, Neisseria meningitidis
MKKMFLSAVLLLSAAAQTVWADTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFS

SEQ ID NO: 2 - protein, 118 aa, Neisseria meningitidis
DTVFSCKTDNNKYIEVQKINRNLYEYSFGSAAKKEIAIRNSKADLLGRSDRWQGMGSGRWATMKFQNGEFMYTIWTGFDSVTHTESSGVVVERRGKEVARVGCTPKTAQANFNDDDFS

SEQ ID NO: 3 - DNA, 420 bp, Neisseria meningitidis
atgaaaaaaatgttcctttctgccgtattgcttctgtcggctgccgcccaaaccgtgtgggcggatacggtgttttcctgtaaaacggacaacaacaaatacatagaagtccaaaaaatcaaccgcaatctttacgaatattcgttcggcagtgcggcaaaaaaagaaattgccatacgcaacagcaaagctgacctgttggggcgttccgacaggtggcaaggtatgggcagcggtcgttgggcaacgatgaaattccaaaacggcgaatttatgtacaccatatggacaggcttcgattccgtgactcatacggaaagcagcggtgtcgttgtggagcgtaggggcaaggaagtcgcacgggtcggctgtacgccgaaaaccgcgcaggcgaatttcaacgatgacgatttttcctag

SEQ ID NO: 4 - DNA, 360 bp, Neisseria meningitidis
atggatacggtgttttcctgtaaaacggacaacaacaaatacatagaagtccaaaaaatcaaccgcaatctttacgaatattcgttcggcagtgcggcaaaaaaagaaattgccatacgcaacagcaaagctgacctgttggggcgttccgacaggtggcaaggtatgggcagcggtcgttgggcaacgatgaaattccaaaacggcgaatttatgtacaccatatggacaggcttcgattccgtgactcatacggaaagcagcggtgtcgttgtggagcgtaggggcaaggaagtcgcacgggtcggctgtacgccgaaaaccgcgcaggcgaatttcaacgatgacgatttttcctag

SEQ ID NO: 5 - protein, 140 aa, Neisseria gonorrhoeae
MKKMFLSAALLLSAAAQTVWADTVFSCKTDNNKYIEVQKINRNLYEYSFGSAAKKEIAIRNSKADLLGRSDRWQGMGSGRRATMKFQNGEFMYTVWTGFDSVTHTESSGVVVERRGKEVARVGCTPKTAQANFNDDDFSR

SEQ ID NO: 6 - protein, 119 aa, Neisseria gonorrhoeae
DTVFSCKTDNNKYIEVQKINRNLYEYSFGSAAKKEIAIRNSKADLLGRSDRWQGMGSGRRATMKFQNGEFMYTVWTGFDSVTHTESSGVVVERRGKEVARVGCTPKTAQANFNDDDFSR

SEQ ID NO: 7 - DNA, 423 bp, Neisseria gonorrhoeae
atgaaaaaaatgttcctttctgccgcattgcttctgtcggctgccgcccaaaccgtgtgggcggatacggtattttcctgtaaaacggacaacaacaaatacatagaagttcaaaaaatcaaccgcaatctttacgaatattcgttcggcagtgcggcaaaaaaagaaattgccatacgcaacagcaaagctgacctgttggggcgttccgacaggtggcaaggcatgggcagcggacgccgggcaacgatgaaattccaaaacggcgaatttatgtacaccgtatggacaggcttcgattccgtgactcatacggaaagcagcggtgtcgttgtggagcgtaggggcaaggaagtcgcacgggtaggctgtacgccgaaaaccgcgcaggcgaatttcaacgatgacgatttttcccggtaa

SEQ ID NO: 8 - DNA, 363 bp, Neisseria gonorrhoeae
atggatacggtattttcctgtaaaacggacaacaacaaatacatagaagttcaaaaaatcaaccgcaatctttacgaatattcgttcggcagtgcggcaaaaaaagaaattgccatacgcaacagcaaagctgacctgttggggcgttccgacaggtggcaaggcatgggcagcggacgccgggcaacgatgaaattccaaaacggcgaatttatgtacaccgtatggacaggcttcgattccgtgactcatacggaaagcagcggtgtcgttgtggagcgtaggggcaaggaagtcgcacgggtaggctgtacgccgaaaaccgcgcaggcgaatttcaacgatgacgatttttcccggtaa

SEQ ID NO: 9 - protein, 140 aa, Neisseria cinerea
MKRTLILIFSGMIGTAGIAQAGSVFSCKTDNNKYIEVQKINRNLYEYSFGSAAKKEIAIRNSKADLLGRSDRWQGIGRGRWATMKFQNGEFMYTVWTDFDSVTHTESSGVVVERRGKEVARVGCTPKTAQANFDDDDFSW

SEQ ID NO: 10 - protein, 119 aa, Neisseria cinerea
GSVFSCKTDNNKYIEVQKINRNLYEYSFGSAAKKEIAIRNSKADLLGRSDRWQGIGRGRWATMKFQNGEFMYTVWTDFDSVTHTESSGVVVERRGKEVARVGCTPKTAQANFDDDDFSW

SEQ ID NO: 11 - DNA, 423 bp, Neisseria cinerea
atgaaaagaaccttgattttgattttttcaggaatgattggcacggcaggtattgcccaagcaggttcggtattttcctgtaagacggacaacaacaaatatatagaagttcaaaaaatcaaccgcaatctttacgaatattcgttcggcagtgcggcaaaaaaagaaattgccatacgcaacagcaaagctgacctgttggggcgttccgacaggtggcaaggcataggtagaggacgctgggcaacgatgaaattccaaaacggcgaatttatgtacaccgtatggacagacttcgattccgtgactcatacggaaagcagcggtgtcgttgtggagcgtaggggcaaggaagtcgcacgggtcggctgtacaccgaaaaccgcgcaggcaaatttcgatgatgacgacttttcttggtaa

SEQ ID NO: 12 - DNA, 363 bp, Neisseria cinerea
atgggttcggtattttcctgtaagacggacaacaacaaatatatagaagttcaaaaaatcaaccgcaatctttacgaatattcgttcggcagtgcggcaaaaaaagaaattgccatacgcaacagcaaagctgacctgttggggcgttccgacaggtggcaaggcataggtagaggacgctgggcaacgatgaaattccaaaacggcgaatttatgtacaccgtatggacagacttcgattccgtgactcatacggaaagcagcggtgtcgttgtggagcgtaggggcaaggaagtcgcacgggtcggctgtacaccgaaaaccgcgcaggcaaatttcgatgatgacgacttttcttggtaa

SEQ ID NO: 13 - protein, 152 aa, Providencia alcalifaciens
MKLRRTSIAIVLPILMSAPVLADSHTVTVFQCVNKNDRQVKVTLINGQYQYQFGKLNQQPDIEITRSPKQLTQGFFQTRYADTDTGYAASQEIDFRNGAYTYTIFTNHSGSKIESGVEVYNKDKRLTKVVCVPNTVVDNIYEHLSEIPERPE

SEQ ID NO: 14 - protein, 130 aa, Providencia alcalifaciens
DSHTVTVFQCVNKNDRQVKVTLINGQYQYQFGKLNQQPDIEITRSPKQLTQGFFQTRYADTDTGYAASQEIDFRNGAYTYTIFTNHSGSKIESGVEVYNKDKRLTKVVCVPNTVVDNIYEHLSEIPERPE

SEQ ID NO: 15 - DNA, 459 bp, Providencia alcalifaciens
atgaaacttcgccgtacatctatagctatagtattaccgatattaatgtccgcccccgtattggcagattcacatacagtgactgtatttcaatgcgttaacaagaatgaccgtcaagtcaaagtgacattgattaatggtcagtatcagtatcaatttggaaaattaaatcagcaaccggacattgaaattacccgaagtcctaaacagcttacgcagggctttttccaaacccgttatgccgatactgatactggatatgcggcttctcaagaaattgattttagaaatggggcttatacctataccatttttacaaaccattcaggctctaaaatagaatcaggtgttgaggtgtataacaaagataagagactgacaaaggttgtttgtgtgccaaatactgttgttgataatatttatgagcacctttcagagatccctgaacgtcctgaataa

SEQ ID NO: 16 - DNA, 396 bp, Providencia alcalifaciens
atggattcacatacagtgactgtatttcaatgcgttaacaagaatgaccgtcaagtcaaagtgacattgattaatggtcagtatcagtatcaatttggaaaattaaatcagcaaccggacattgaaattacccgaagtcctaaacagcttacgcagggctttttccaaacccgttatgccgatactgatactggatatgcggcttctcaagaaattgattttagaaatggggcttatacctataccatttttacaaaccattcaggctctaaaatagaatcaggtgttgaggtgtataacaaagataagagactgacaaaggttgtttgtgtgccaaatactgttgttgataatatttatgagcacctttcagagatccctgaacgtcctgaataa

SEQ ID NO: 17 - DNA, 459 bp, Providencia alcalifaciens; identical to SEQ ID NO: 15

SEQ ID NO: 18 - protein, 149 aa, Proteus penneri
MKARIAIAAVFVLLSASVYAEEFTCQLENGKYVSVFVEHGKPPVYRYGTLAKTEITLPVPQQPNNNVFYGHQMFVAGASTYIRFKNGNYSYIVYDGEGRGWNFNGIIVYKDNNIISKKECKQPLTPSLDKLKNYSIKMDPYLETYIYAP

SEQ ID NO: 19 - protein, 129 aa, Proteus penneri
EEFTCQLENGKYVSVFVEHGKPPVYRYGTLAKTEITLPVPQQPNNNVFYGHQMFVAGASTYIRFKNGNYSYIVYDGEGRGWNFNGIIVYKDNNIISKKECKQPLTPSLDKLKNYSIKMDPYLETYIYAP

SEQ ID NO: 20 - DNA, 450 bp, Proteus penneri
atgaaagcacgtatcgctattgctgctgtattcgtattattatccgcaagtgtctatgctgaagaatttacctgccaacttgaaaatggtaagtatgtcagcgtgttcgttgaacatggaaagccacctgtttatcgctatggcacacttgcaaaaacagagatcacactacctgttcctcagcaacccaataataatgtcttttatgggcatcaaatgtttgttgctggcgcatcgacttatattcgatttaagaatggaaattacagctacattgtttatgacggtgaaggtagaggatggaactttaacggtataattgtttataaagacaataacattatcagtaaaaaagagtgcaaacagccattaacacccagcttagataagttaaaaaattactcaattaaaatggacccttatttagaaacctatatttatgctccttaa

SEQ ID NO: 21 - DNA, 393 bp, Proteus penneri
atggaagaatttacctgccaacttgaaaatggtaagtatgtcagcgtgttcgttgaacatggaaagccacctgtttatcgctatggcacacttgcaaaaacagagatcacactacctgttcctcagcaacccaataataatgtcttttatgggcatcaaatgtttgttgctggcgcatcgacttatattcgatttaagaatggaaattacagctacattgtttatgacggtgaaggtagaggatggaactttaacggtataattgtttataaagacaataacattatcagtaaaaaagagtgcaaacagccattaacacccagcttagataagttaaaaaattactcaattaaaatggacccttatttagaaacctatatttatgctccttaa

SEQ ID NO: 22 - protein, 153 aa, Providencia rustigianii
MYKKIAVCLALGLFTASASAVEYTCQLENNKTVSVVVETGKVPVYRYGTLGKTEITLPINAKGQESIYVGQGTFSAGGSIYIRFQNGPFSYLLYDGEGRGWYFQGIAVYKNGKLVNKKSCKPDSNLNLYSLLDYGIPTDPDEDGPYTFDPNSN

SEQ ID NO: 23 - protein, 133 aa, Providencia rustigianii
VEYTCQLENNKTVSVVVETGKVPVYRYGTLGKTEITLPINAKGQESIYVGQGTFSAGGSIYIRFQNGPFSYLLYDGEGRGWYFQGIAVYKNGKLVNKKSCKPDSNLNLYSLLDYGIPTDPDEDGPYTFDPNSN

SEQ ID NO: 24 - DNA, 462 bp, Providencia rustigani
atgtataagaaaattgccgtttgtttagcactaggattatttactgcatctgctagtgcagttgaatatacttgccagctagaaaataacaaaacggtcagcgtcgtcgttgagacaggaaaagtgcctgtttatcgctatgggactttaggtaaaactgaaataacacttccaatcaatgcgaaagggcaagaatctatttatgtagggcaaggaacattcagcgctggtggctcgatatatatccgtttccaaaatggaccatttagctatttgctttatgatggggaaggtaggggctggtattttcaggggattgctgtttataaaaacggtaagctggttaataagaaatcgtgcaaacctgactctaatttaaatctgtatagcctgctagactacggtattccaactgatcctgatgaagatggaccttatactttcgatccaaattcaaattaa

SEQ ID NO: 25 - DNA, 405 bp, Providencia rustigianii
atggttgaatatacttgccagctagaaaataacaaaacggtcagcgtcgtcgttgagacaggaaaagtgcctgtttatcgctatgggactttaggtaaaactgaaataacacttccaatcaatgcgaaagggcaagaatctatttatgtagggcaaggaacattcagcgctggtggctcgatatatatccgtttccaaaatggaccatttagctatttgctttatgatggggaaggtaggggctggtattttcaggggattgctgtttataaaaacggtaagctggttaataagaaatcgtgcaaacctgactctaatttaaatctgtatagcctgctagactacggtattccaactgatcctgatgaagatggaccttatactttcgatccaaattcaaattaa

SEQ ID NO: 26 - protein, 157 aa, Providencia stuartii
MNKTLLGMVIIGVSATPVMAAEFTCQLENKKYVSVIVDPGKTPQYRYGTLAKPEITLPLNEKGARNIFVGQGHFIAGESLYIRFQNGPYSYLLYQGEGRGWAFKGLAVYKNNKLINKKECQNWNSFDLYDILNYQIRTDPALELYDSPYSFDPNSNE

SEQ ID NO: 27 - protein, 137 aa, Providencia stuartii
AEFTCQLENKKYVSVIVDPGKTPQYRYGTLAKPEITLPLNEKGARNIFVGQGHFIAGESLYIRFQNGPYSYLLYQGEGRGWAFKGLAVYKNNKLINKKECQNWNSFDLYDILNYQIRTDPALELYDSPYSFDPNSNE

SEQ ID NO: 28 - DNA, 474 bp, Providencia stuartii
atgaataaaacattacttgggatggttataattggggtatctgcaacaccagtgatggccgcagaatttacctgccaactggagaataaaaagtatgttagcgtgatagtcgacccggggaaaacaccacaatatcgatacggaactttggctaagccagagataaccttaccgcttaatgaaaaaggagcgagaaatatttttgttgggcaaggacattttattgctggtgaatctctatatattcgttttcaaaatggcccatacagctatttgttgtatcaaggcgaggggcgcggctgggcatttaagggattagctgtttataaaaataacaagttaatcaataagaaagagtgccaaaattggaattcatttgatttatacgacattttaaattatcagattcgtacagatccagcattagaattatatgattccccttactcatttgatcctaattcaaatgagtaa

SEQ ID NO: 29 - DNA, 417 bp, Providencia stuartii
atggcagaatttacctgccaactggagaataaaaagtatgttagcgtgatagtcgacccggggaaaacaccacaatatcgatacggaactttggctaagccagagataaccttaccgcttaatgaaaaaggagcgagaaatatttttgttgggcaaggacattttattgctggtgaatctctatatattcgttttcaaaatggcccatacagctatttgttgtatcaaggcgaggggcgcggctgggcatttaagggattagctgtttataaaaataacaagttaatcaataagaaagagtgccaaaattggaattcatttgatttatacgacattttaaattatcagattcgtacagatccagcattagaattatatgattccccttactcatttgatcctaattcaaatgagtaa

SEQ ID NO: 30 - protein, 138 aa, Vibrio cholerae
MSIFTLFSSILLMSLHVNAQTIFYCETQNNKKVEVQIVGDLLQYRFGEKLYKPEIEVLVARRKTSTFQWLGVGMDEYYDVTIPNRDVFYKVFTSRERKPEGKLEAGISVWSKDKLIAEIYCNNDSLYEVLFDIELPKE

SEQ ID NO: 31 - protein, 119 aa, Vibrio cholerae
QTIFYCETQNNKKVEVQIVGDLLQYRFGEKLYKPEIEVLVARRKTSTFQWLGVGMDEYYDVTIPNRDVFYKVFTSRERKPEGKLEAGISVWSKDKLIAEIYCNNDSLYEVLFDIELPKE

SEQ ID NO: 32 - DNA, 417 bp, Vibrio cholerae
atgtcaatatttacacttttttcatcgatacttttgatgtcattacatgtaaatgctcagacaattttttactgcgaaacgcagaacaataaaaaagttgaagtacagatagttggtgatttattgcaataccgttttggtgagaagctatataaacctgagattgaggtgcttgttgcaagaagaaaaacttcaacatttcagtggttgggcgttggaatggatgagtattatgatgtaacaattccaaaccgagacgttttttataaggtgtttacatcccgagaaagaaagcctgaaggtaagttagaagctggtatcagtgtttggtcaaaagataagttaattgctgaaatatactgcaacaatgactcattgtatgaggtgttgtttgatattgaactacccaaggaatga

SEQ ID NO: 33 - DNA, 363 bp, Vibrio cholerae
atgcagacaattttttactgcgaaacgcagaacaataaaaaagttgaagtacagatagttggtgatttattgcaataccgttttggtgagaagctatataaacctgagattgaggtgcttgttgcaagaagaaaaacttcaacatttcagtggttgggcgttggaatggatgagtattatgatgtaacaattccaaaccgagacgttttttataaggtgtttacatcccgagaaagaaagcctgaaggtaagttagaagctggtatcagtgtttggtcaaaagataagttaattgctgaaatatactgcaacaatgactcattgtatgaggtgttgtttgatattgaactacccaaggaatga

SEQ ID NO: 34 - protein, 140 aa, Pseudomonas mendocina
MMRIFFVLVFSICFPLVVNGSESHMPIFMCKTKNNKNIEVYRDGDMVTYSFGRSDYPPELKLTRSMADLEVAIGSVSGNEISNSITFSNGVYAYRVMTTINKVSATQEPRHGVLVKKKTKYLAYIDCVPETVQGSLLDLE

SEQ ID NO: 35 - protein, 120 aa, Pseudomonas mendocina
SESHMPIFMCKTKNNKNIEVYRDGDMVTYSFGRSDYPPELKLTRSMADLEVAIGSVSGNEISNSITFSNGVYAYRVMTTINKVSATQEPRHGVLVKKKTKYLAYIDCVPETVQGSLLDLE

SEQ ID NO: 36 - DNA, 423 bp, Pseudomonas mendocina
atgatgcgtatattttttgtcttggtcttttctatatgctttccgttagtggtaaatggctcggaaagccatatgcctatttttatgtgcaaaacgaaaaataataagaatattgaggtgtatagagatggagatatggtgacctattcgtttggtagaagcgattacccgccagaacttaaattgacacggtcaatggcagatttagaggtcgcaataggcagtgtttctgggaatgaaatttcgaattctataactttttcaaatggcgtgtatgcgtacagagtgatgacaacgattaataaagtgtctgcgacgcaggagcccaggcatggggtcttggtcaagaaaaaaactaagtacttagcctatatagactgcgttccagaaactgtgcaaggaagtttgcttgacttggagtga

SEQ ID NO: 37 - DNA, 366 bp, Pseudomonas mendocina
atgtcggaaagccatatgcctatttttatgtgcaaaacgaaaaataataagaatattgaggtgtatagagatggagatatggtgacctattcgtttggtagaagcgattacccgccagaacttaaattgacacggtcaatggcagatttagaggtcgcaataggcagtgtttctgggaatgaaatttcgaattctataactttttcaaatggcgtgtatgcgtacagagtgatgacaacgattaataaagtgtctgcgacgcaggagcccaggcatggggtcttggtcaagaaaaaaactaagtacttagcctatatagactgcgttccagaaactgtgcaaggaagtttgcttgacttggagtga

SEQ ID NO: 38 - protein, 135 aa, Haemophilus influenzae
MKKLLTIGAVAMFATPAFAANNIFSCTAENGSPVSVTKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKAS

SEQ ID NO: 39 - protein, 116 aa, Haemophilus influenzae
ANNIFSCTAENGSPVSVTKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKAS

SEQ ID NO: 40 - DNA, 408 bp, Haemophilus influenzae
atgaaaaaattactcactattggagcggtagctatgtttgcaacgccagcttttgcagctaataatattttttcttgtacagcagagaacggaagcccggtgtctgtaacaaaaaatggttcagattatgaattcacttatggccaggtttcttttaaaaatccggtaaaacaagtttttgctaatcaggattcttatgtagctactggttcaggatttattactagctcattagaaatgagaaataatggcacaagttacacgattcaatttgtgcaaccacacaatagtaacagtattgaagagccaatgctttatatcaccaatggcagtaaaatggatacggtgagctgtaaagcgggtagtgcaactcaaaactttgaacgtcgcagtatgaaagcttcataa

SEQ ID NO: 41 - DNA, 354 bp, Haemophilus influenzae
atggctaataatattttttcttgtacagcagagaacggaagcccggtgtctgtaacaaaaaatggttcagattatgaattcacttatggccaggtttcttttaaaaatccggtaaaacaagtttttgctaatcaggattcttatgtagctactggttcaggatttattactagctcattagaaatgagaaataatggcacaagttacacgattcaatttgtgcaaccacacaatagtaacagtattgaagagccaatgctttatatcaccaatggcagtaaaatggatacggtgagctgtaaagcgggtagtgcaactcaaaactttgaacgtcgcagtatgaaagcttcataa

SEQ ID NO: 42 - protein, 135 aa, Haemophilus parainfluenzae
MKKLLTIAAVAVCATPAFAANNIFSCTAENGSPVSVVKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKVS

SEQ ID NO: 43 - protein, 116 aa, Haemophilus parainfluenzae
ANNIFSCTAENGSPVSVVKNGSDYEFTYGQVSFKNPVKQVFANQDSYVATGSGFITSSLEMRNNGTSYTIQFVQPHNSNSIEEPMLYITNGSKMDTVSCKAGSATQNFERRSMKVS

SEQ ID NO: 44 - DNA, 408 bp, Haemophilus parainfluenzae
atgaaaaaattactcactattgcagcggttgctgtgtgtgcaacacctgcatttgcagctaataatattttttcttgtacggcagagaacggaagcccggtatctgtagtaaagaatggttcagactatgaatttacttatggccaagtttcttttaaaaatccggtaaaacaagtttttgctaatcaggattcttatgtagctactggttcagggtttattactagctcattagaaatgagaaataatggcacaagttacacgattcaatttgtgcaaccacacaatagtaacagtattgaagagccaatgctttatatcaccaatggcagtaaaatggatacggtgagctgtaaagcgggtagtgcaactcaaaactttgaacgtcgcagtatgaaagtttcataa

SEQ ID NO: 45 - DNA, 354 bp, Haemophilus parainfluenzae
atggctaataatattttttcttgtacggcagagaacggaagcccggtatctgtagtaaagaatggttcagactatgaatttacttatggccaagtttcttttaaaaatccggtaaaacaagtttttgctaatcaggattcttatgtagctactggttcagggtttattactagctcattagaaatgagaaataatggcacaagttacacgattcaatttgtgcaaccacacaatagtaacagtattgaagagccaatgctttatatcaccaatggcagtaaaatggatacggtgagctgtaaagcgggtagtgcaactcaaaactttgaacgtcgcagtatgaaagtttcataa

SEQ ID NO: 46 - protein, 176 aa, Cronobacter turicensis
MVALWGLLREEMLTNYVKRFFYEIYIFYFFIMFFYNCHAAGCNVEENLVASCDLPGQIKRTAAFCADKNNKIKYYFKESHAIKFKIEFNSERKLKRWLDLGTYTTYFGFNNASYTYVLGVPEEKPGALAFLEIKKNGKTISSKECSSNSFGEKNIESDSIEDVPDSVVRGDFFKFP

SEQ ID NO: 47 - protein, 137 aa, Cronobacter turicensis
AGCNVEENLVASCDLPGQIKRTAAFCADKNNKIKYYFKESHAIKFKIEFNSERKLKRWLDLGTYTTYFGFNNASYTYVLGVPEEKPGALAFLEIKKNGKTISSKECSSNSFGEKNIESDSIEDVPDSVVRGDFFKFP

SEQ ID NO: 48 - DNA, 531 bp, Cronobacter turicensis
atggtggcgctatggggcttgctgagagaagagatgcttacaaattatgtgaaaaggtttttttatgaaatttatattttttattttttcattatgtttttttataattgccatgctgctggatgcaacgttgaagaaaatcttgtggcgtcatgtgaccttccgggtcaaataaaacggaccgctgcattttgtgctgataaaaataacaaaattaaatattattttaaagagtctcatgctatcaaatttaaaatagaattcaactctgaaaggaagttaaaaagatggttggatcttggaacttataccacatactttggttttaataatgcatcttatacttatgttcttggggttcctgaagaaaaacccggagccttggcatttcttgaaatcaaaaaaaacgggaagactataagctctaaagaatgctcttctaattcatttggtgagaaaaatattgaaagtgacagtattgaggatgtaccagattcagtggtgaggggtgatttttttaaatttccttaa

SEQ ID NO: 49 - DNA, 417 bp, Cronobacter turicensis
atggctggatgcaacgttgaagaaaatcttgtggcgtcatgtgaccttccgggtcaaataaaacggaccgctgcattttgtgctgataaaaataacaaaattaaatattattttaaagagtctcatgctatcaaatttaaaatagaattcaactctgaaaggaagttaaaaagatggttggatcttggaacttataccacatactttggttttaataatgcatcttatacttatgttcttggggttcctgaagaaaaacccggagccttggcatttcttgaaatcaaaaaaaacgggaagactataagctctaaagaatgctcttctaattcatttggtgagaaaaatattgaaagtgacagtattgaggatgtaccagattcagtggtgaggggtgatttttttaaatttccttaa

SEQ ID NO: 50 - protein, 173 aa, Burkholderia spp
MPRDRIAELIVALSMLNVAHAEGLCRSSETTVFNCDLKENHKIVSICSSKDLTDKSGFLQYRYGSRNKVEFAFPKSLTNSQSQFGYDEYSRPDLSTFVVGFNYENYRYEISETTEGSDDDALTTRTLLVSSGTTTRGLKLTCLDNQNTVSDISTLDHVLKCDKKPAIIEGACD

SEQ ID NO: 51 - protein, 152 aa, Burkholderia spp
EGLCRSSETTVFNCDLKENHKIVSICSSKDLTDKSGFLQYRYGSRNKVEFAFPKSLTNSQSQFGYDEYSRPDLSTFVVGFNYENYRYEISETTEGSDDDALTTRTLLVSSGTTTRGLKLTCLDNQNTVSDISTLDHVLKCDKKPAIIEGACD

SEQ ID NO: 52 - DNA, 522 bp, Burkholderia spp
atgccgagagacagaattgcggagttgatcgtcgccctttcgatgctgaatgtggcgcatgcagaagggctatgtcgctcgagcgagacgaccgtgttcaactgcgatctgaaagaaaatcacaagatcgtatcaatatgctcgtcgaaggatctgacagataagagcggatttttgcaatatcggtatggttcgcgaaacaaggtcgaatttgcattcccgaagagcttgactaactcgcagtcgcagttcggctacgacgaatattctcgccctgatttgagcacgttcgtggtgggcttcaattacgagaactatcgctacgagatatcagagaccactgagggtagcgacgacgacgccctcacgacccgcacgctactggtgagctcaggcacaacgacgcgaggcctgaagctgacgtgtctcgataaccagaacacagtctctgacatttcgacgctcgaccacgtgctgaagtgcgacaagaagcccgcgatcatcgagggtgcgtgcgactaa

SEQ ID NO: 53 - DNA, 462 bp, Burkholderia spp
atggaagggctatgtcgctcgagcgagacgaccgtgttcaactgcgatctgaaagaaaatcacaagatcgtatcaatatgctcgtcgaaggatctgacagataagagcggatttttgcaatatcggtatggttcgcgaaacaaggtcgaatttgcattcccgaagagcttgactaactcgcagtcgcagttcggctacgacgaatattctcgccctgatttgagcacgttcgtggtgggcttcaattacgagaactatcgctacgagatatcagagaccactgagggtagcgacgacgacgccctcacgacccgcacgctactggtgagctcaggcacaacgacgcgaggcctgaagctgacgtgtctcgataaccagaacacagtctctgacatttcgacgctcgaccacgtgctgaagtgcgacaagaagcccgcgatcatcgagggtgcgtgcgactaa

SEQ ID NO: 54 - DNA, 357 bp, Moraxella catarrhalis
atgaaaaaacttgcctttagtcttattgtggctactgctggcatgatggctatttctgccaatgccatgagtccaaaaaccgtacaatatacctgccaaggcggtaaaagtgtcaatgttaaatacatctttaacgatgcagatttaccatctaaagccgttgctagcttctctggcaaaactgtcggtatgcccatcaatcttaacgcatcagatatgacaagctcaatttttggctttggtggttataccatgactgcagactatatcgatgccaagaactacaatcaagtcggtattgcagtcatcaccgacccaaagaacaaaactttgttcaaaaactgcaacccaagataa

SEQ ID NO: 55 - protein, 118 aa, Moraxella catarrhalis
MKKLAFSLIVATAGMMAISANAMSPKTVQYTCQGGKSVNVKYIFNDADLPSKAVASFSGKTVGMPINLNASDMTSSIFGFGGYTMTADYIDAKNYNQVGIAVITDPKNKTLFKNCNPR

SEQ ID NO: 56 - protein, 96 aa, Moraxella catarrhalis
MSPKTVQYTCQGGKSVNVKYIFNDADLPSKAVASFSGKTVGMPINLNASDMTSSIFGFGGYTMTADYIDAKNYNQVGIAVITDPKNKTLFKNCNPR

SEQ ID NO: 57 - DNA, 369 bp, Eikenella corrodens
atgaaactattatctgttgttactgcggctgcactggctgccgtttccctgagtgctgcggctgcgcccggcggcatggtttcctacacctgcgacaacggcaaacagttgaacgtgttgtatgagttcaaccgccaaggcaagccggtaagcgctgcggtgaatgccgccggtacgcaggtaaacctggcttataacagaagacagtccgacagcaccggtaccaccttcagcaaccgtcgcggctactctctgtctgccggctacatcgacagaaacacccacaccacctccgatgtggtgggtttgaccgctcctggcggccgtttcgtggtgaaaaactgctcacctgtgaacgccagcaactaa

SEQ ID NO: 58 - protein, 122 aa, Eikenella corrodens
MKLLSVVTAAALAAVSLSAAAAPGGMVSYTCDNGKQLNVLYEFNRQGKPVSAAVNAAGTQVNLAYNRRQSDSTGTTFSNRRGYSLSAGYIDRNTHTTSDVVGLTAPGGRFVVKNCSPVNASN

SEQ ID NO: 59 - protein, 101 aa, Eikenella corrodens
APGGMVSYTCDNGKQLNVLYEFNRQGKPVSAAVNAAGTQVNLAYNRRQSDSTGTTFSNRRGYSLSAGYIDRNTHTTSDVVGLTAPGGRFVVKNCSPVNASN

SEQ ID NO: 60 - DNA, 411 bp, Aggregatibacter actinomycetemcomitans
atgaaattagtaaaaacattatctgctgttgcagtcattgcggtactgtccgcgtgttctgcttccaagaaagaaccacaaccggttaaatcggagaaaactgtggtttattcttgtaacaaaagagccgttaccgtgacttaccaatttgaaaaccaagaagccactgaagcgaaagttgtgatgcgcaaaaaggtgattgcggaatctcttccggtgagtaacgtcgataaagatttcccttcatttatttccgacaaatacatctggagcgttgacagcgggttttcattaaatacggcaacagcaaccgatggcgtaatgttgacccaacgcggtaagaaagtggatagaatcttggctaaaaattgtaaagtcaatgcaaaagccacagcaagagctaaccaatag

SEQ ID NO: 61 - protein, 136 aa, Aggregatibacter actinomycetemcomitans
MKLVKTLSAVAVIAVLSACSASKKEPQPVKSEKTVVYSCNKRAVTVTYQFENQEATEAKVVMRKKVIAESLPVSNVDKDFPSFISDKYIWSVDSGFSLNTATATDGVMLTQRGKKVDRILAKNCKVNAKATARANQ

SEQ ID NO: 62 - protein, 113 aa, Aggregatibacter actinomycetemcomitans
KEPQPVKSEKTVVYSCNKRAVTVTYQFENQEATEAKVVMRKKVIAESLPVSNVDKDFPSFISDKYIWSVDSGFSLNTATATDGVMLTQRGKKVDRILAKNCKVNAKATARANQ

SEQ ID NO: 63 - DNA, 786 bp, Escherichia coli
atgaaactaatcattatacctgcattgtttgtatctttatttgcattccgccctgcttatgcagtcggggcctatgacctgcaatgtatgcttgataacggcgagcaaatgacactcagccatgtcagcaataccgtttatatcagttttgaaaatcctggcggtgactccgaagagagcggctctgttattaaactggatattccttccggcgaagcgaaacagactctggcggctaatccgggagcaggaaccgcttctttcacattacgtggagaaaatgaagatatcgaaggcgcggttgcagtcaattactcagaatatgatggtacaggtgatgcttattacactgtcatgaatgcaatggggcaggaaaccagcacagtatcatgcaagccgggtaccattaaggtttccagaagcctccttcagaatggcattaatagcgttggtagccagcaggctaataaacctgctccctctcaacaacagcaggcacagcaatctacgacaccaccatttaaagttcagttcggcagctccgtcagtaatgaaggctggaataccaaatatggtgttattcagctcactttcactgatgacaacgtggtgctgaaatccattcgggtaaacagagggaactgtaaaatggagagcgtcggtaacagaaccctgccagcaaaatataaattcggtgatgtggctacgttcaaatatatgaagtgtgaccgtatcattgaagctgacattgtgaccgatacgggtagctggacatttaattcctaa

SEQ ID NO: 64 - protein, 265 aa, Escherichia coli
ETECMKLIIIPALFVSLFAFRPAYAVGAYDLQCMLDNGEQMTLSHVSNTVYISFENPGGDSEESGSVIKLDIPSGEAKQTLAANPGAGTASFTLRGENEDIEGAVAVNYSEYDGTGDAYYTVMNAMGQETSTVSCKPGTIKVSRSLLQNGINSVGSQQANKPAPSQQQQAQQSTTPPFKVQFGSSVSNEGWNTKYGVIQLTFTDDNVVLKSIRVNRGNCKMESVGNRTLPAKYKFGDVATFKYMKCDRIIEADIVTDTGSWTFNS

SEQ ID NO: 65 - DNA, 528 bp, Kingella denitrificans
atgcgcgccaactgcggcacgatgattttcaagcagcataaccgtgcaaaaagcagcctgcacttaaaaaaaggacaccccatgcccgccctaatccccgcattcgccgcactcctgctcgccagcactcccgtctctgccaccgctgacacgctgttttattgccaaatcgccaacggcaagcaagtgcatatccggcaacatgacggcaacgtgcgctaccgctttggcaaaaacctggcccgcccagaactggaatttacacaacctaaaacacaggtttaccagcgaaactttacgatcgaagacaacggcgaccgtgcccacgcaagacaaatgtggctgcgcaacggcaatgcggactatgtcaccaccgtgctttacctttcagaaggaagagcgtatttgctgggcgtataccatgacggcgaatatgccgaaattccctgccggcagcgaaacgcctttgaaaaaataacgctcatcgatgccctgcctgaacttccagacgattttcaaactgaataa

SEQ ID NO: 66 - protein, 154 aa, Kingella denitrificans
MKKHILFTNHPKYKECPMKKITLITLLFFVSAATANNVVEDIDYQQEEPIFYCKAPNKKIIQLTKYEEVYIYRFGKNLDKPEIRLENHIEDVKRTSETQNGFETKRAYELTTGPYIYRIFSGIPKVPEENGVEVFKHRKRIARIECSGYVHIFE

SEQ ID NO: 67 - DNA, 465 bp, Pasteurella dagmatis
atgaaaaagcatatactatttacaaatcatccaaaatataaggaatgccctatgaaaaaaataacactcattaccttattattctttgttagtgctgctacagccaataatgttgtagaagatatagactatcaacaagaagaacccattttctactgtaaagcgcctaacaaaaaaattattcaattaaccaaatatgaggaagtctatatttatcgtttcggtaaaaaccttgataaaccagagattagactcgaaaatcatattgaagatgtaaaacgaaccagtgaaactcaaaacggatttgaaactaaacgtgcctatgagttaacaactgggccttatatttatcgtatattttctggtattcctaaagttcctgaagagaatggtgttgaagtattcaaacatagaaaacgaattgcaaggatcgaatgttctggatatgtccatatttttgagtga

SEQ ID NO: 68 - protein, 154 aa, Pasteurella dagmatis; identical to SEQ ID NO: 66

SEQ ID NO: 69 - protein, 148 aa, Salmonella enterica
MHHYKFFALAAMLLCGSSFASDEYFMCNTAKGTIKLDENKGVLRYTLSKDRKTAFNYESKGNDYSGFKYNHYSRFQTDYFSVSFVNAEYKYTIFSNYEGESESRGVSVTNLNSKKESVYDCKSVSIDRLSDLSAKLACDKDSALGCEE

SEQ ID NO: 70 - DNA, 483 bp, Salmonella enterica
gaattctaccgaggacttaattaaataatgcatcattataagttttttgcgttggctgcaatgcttttgtgtggttcatcgtttgcgagtgatgaatatttcatgtgcaacactgcaaaaggaacaatcaaactggatgaaaacaaaggcgtgcttcggtacaccttatcgaaagaccgcaaaactgcatttaattatgaatcgaaaggtaatgattattcaggatttaaatacaatcattactcacgatttcaaaccgattatttcagtgtatcgtttgtaaatgcagagtataagtacacgatctttagtaattatgagggtgagagtgaaagccgtggagttagtgttacgaacctgaatagcaaaaaggaatctgtttacgattgtaagtccgttagcattgatcgtcttagtgatttgtcggcaaaactggcttgtgataaagattctgctttggggtgtgaagaatgataaaagctt

SEQ ID NO: 71 - protein, 163 aa, Salmonella enterica
MHHYKFFALAAMLLCGSSFASDEYFMCNTAKGTIKLDENKGVLRYTLSKDRKTAFNYESKGNDYSGFKYNHYSRFQTDYFSVSFVNAEYKYTIFSNYEGESESRGVSVTNLNSKKESVYDCKSVSIDRLSDLSAKLACDKDSALGCEEGILQDDDDKHHHHHH

SEQ ID NO: 72 - DNA, 519 bp, Salmonella enterica
gaattctaccgaggacttaattaaataatgcatcattataagttttttgcgttggctgcaatgcttttgtgtggttcatcgtttgcgagtgatgaatatttcatgtgcaacactgcaaaaggaacaatcaaactggatgaaaacaaaggcgtgcttcggtacaccttatcgaaagaccgcaaaactgcatttaattatgaatcgaaaggtaatgattattcaggatttaaatacaatcattactcacgatttcaaaccgattatttcagtgtatcgtttgtaaatgcagagtataagtacacgatctttagtaattatgagggtgagagtgaaagccgtggagttagtgttacgaacctgaatagcaaaaaggaatctgtttacgattgtaagtccgttagcattgatcgtcttagtgatttgtcggcaaaactggcttgtgataaagattctgctttggggtgtgaagaagggatcctgcaggatgacgatgacaaacaccatcatcaccatcattag

SEQ ID NO: 73 - DNA, 46 bp, Artificial Sequence
  DNA oligonucleotide
gatcctgcaggatgacgatgacaaacaccatcatcaccatcattag

SEQ ID NO: 74 - DNA, 46 bp, Artificial Sequence
  DNA oligonucleotide
agctctaatgatggtgatgatggtgtttgtcatcgtcatcctgcag

SEQ ID NO: 75 - DNA, 27 bp, Artificial Sequence
  DNA oligonucleotide
tttcgaattctaaggaaatacgatgaa

SEQ ID NO: 76 - DNA, 25 bp, Artificial Sequence
  DNA oligonucleotide
atccgccccgggatcccggaaaaat

SEQ ID NO: 77 - DNA, 53 bp, Artificial Sequence
  DNA oligonucleotide
aataggtacctgaggagaaacaaatgacaaaattactcactcactattggagc

SEQ ID NO: 78 - DNA, 33 bp, Artificial Sequence
  DNA oligonucleotide
tattcgatccctcaagctttcatactgcgacgt

SEQ ID NO: 79 - DNA, 39 bp, Artificial Sequence
  DNA oligonucleotide
aaatgtcgacgcttattttatctctatttacgcttatga

SEQ ID NO: 80 - DNA, 20 bp, Artificial Sequence
  DNA oligonucleotide
gccgacggcaaaacggttca

SEQ ID NO: 81 - DNA, 19 bp, Artificial Sequence
  DNA oligonucleotide
ccaaaccggcaggcgcaaa

SEQ ID NO: 82 - DNA, 33 bp, Artificial Sequence
  DNA oligonucleotide
ggggccctgtgttgatagtgcagtatcttaaaa

SEQ ID NO: 83 - DNA, 36 bp, Artificial Sequence
  DNA oligonucleotide
ggggccctatatctaataatttatctccattccctt

SEQ ID NO: 84 - DNA, 20 bp, Artificial Sequence
  DNA oligonucleotide
gccgacggcaaaacggttca

SEQ ID NO: 85 - DNA, 19 bp, Artificial Sequence
  DNA oligonucleotide
ccaaaccggcaggcgcaaa

(19) United States
(12) Patent Application Publication
(10) Pub. No.: US 2013/0330295 A1
(43) Pub. Date: Dec. 12, 2013

(54) ANTIGENIC GLY1 POLYPEPTIDE

(71) Applicant: Jon Sayers, Sheffield (GB)

(72) Inventor: Jon Sayers, Sheffield (GB)

(21) Appl. No.: 13/981,404

(22) PCT Filed: Feb. 7, 2012
(86) PCT No.: PCT/GB2012/050258

§ 371 Date: Aug. 29, 2013

(30) Foreign Application Priority Data
Feb. 8, 2011 (GB) 1102090.6

(51) Int. Cl. A61K 39/095; A61K 45/06; A61K 39/02

(52) U.S. Cl. CPC A61K 39/095; A61K 39/025; A61K 45/06
USPC 424/85.2; 424/190.1; 424/250.1; 424/234.1; 424/85.1; 424/85.5; 424/85.6; 424/85.7

(57) ABSTRACT

The disclosure relates to antigenic polypeptides that induce the production of opsonins, in particular opsonic antibodies, and the use of said antigenic polypeptides in vaccines that are protective against human bacterial pathogens.

### INTRODUCTION

The disclosure relates to antigenic polypeptides that induce the production of opsonins, in particular opsonic antibodies, and the use of said antigenic polypeptides in vaccines that are protective against human bacterial pathogens.

### BACKGROUND TO THE DISCLOSURE

Pathogenic bacteria are a major cause of infectious diseases that affect many millions of humans. The control of bacterial infection includes the use of antiseptics to control bacterial growth in environments which provide conditions for bacterial growth; the use of antibiotics to control bacterial infections in subjects that are either infected or are susceptible to infection, for example immune suppressed patients; and the development of vaccines that protect the subject from infection [i.e. prophylactic vaccines] or vaccines that are used to treat subjects suffering from infection [i.e. therapeutic vaccines].

Vaccines protect against a wide variety of infectious diseases. Many vaccines are produced by inactivated or attenuated pathogens which are injected into a subject. The immunised subject responds by producing both a humoral (e.g. antibody) and cellular (e.g. cytolytic T cells) responses. For example, some influenza vaccines are made by inactivating the virus by chemical treatment with formaldehyde. For many pathogens chemical or heat inactivation, while it may give rise to vaccine immunogens that confer protective immunity, also gives rise to side effects such as fever and injection site reactions. In the case of bacteria, inactivated organisms tend to be so toxic that side effects have limited the application of such crude vaccine immunogens (e.g. the cellular pertussis vaccine) and therefore vaccine development has lagged behind drug-development. Moreover, effective vaccine development using whole cell inactivated organisms suffers from problems of epitope masking, immunodominance, low antigen concentration and antigen redundancy. This is unfortunate as current antibiotic treatments are now prejudiced by the emergence of drug-resistant bacteria.

Many modern vaccines are therefore made from protective antigens of the pathogen, isolated by molecular cloning and purified from the materials that give rise to side-effects. These vaccines are known as ‘subunit vaccines’. The development of subunit vaccines has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens. However, although subunit vaccines tend to avoid the side effects of killed or attenuated pathogen vaccines, their ‘pure’ status means that subunit vaccines do not always have adequate immunogenicity to confer protection.

As mentioned above vaccines induce the production of antibodies and/or cytolytic T cells that target organisms that express the particular inducing antigen. Antigens that may confer protection tend to be those expressed at the cell surface of the pathogen or alternatively secreted into the surrounding environment and therefore accessible to the immune system. Induced antibodies can function in the process known as opsonisation. Opsonisation is a process by which microbial pathogens are targeted for ingestion by phagocytic cells of the immune system. The binding of opsonins attracts phagocytic cells which results in destruction of the bacterial pathogen. Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fc portion of an antibody or complement component C3.

This disclosure relates to the identification of a class of protective antigen that advantageously induces the production of opsonins that target human bacterial pathogens, for example Nesseria meningitidis. [N. meningitidis]. N. meningitdis is a Gram negative bacterium which is the causative agent of bacterial meningitis and meningococcal disease and septicaemia both of which can cause death, particularly in young children. The onset of meningitis requires immediate medical attention since the progress of the disease is rapid and without intervention can result in coma and death. Typically treatment involves the administration of multiple intravenous antibiotics. It would be highly desirable to provide immunization against this pathogen. The Gly1 antigen is a secreted protein and shown to be essential to the growth of, for example, N. meningitidis when grown in defined media with haem or haemoglobin as the iron source. Gly1 is involved in iron metabolism and provides an essential function since the phenotype of deletion mutants in Gly1 is failure to grow under these conditions. Furthermore, we show that serum from rabbits immunized with Gly1 have bactericidal activity. The Gly1 protein is an example of a class of protein found in many pathogen bacterial species involved in haem sequestration and is essential for maintaining bacterial growth and the establishment of infection.

We disclose vaccine compositions comprising Gly1 and sequence variants thereof and their use in the prophylactic and therapeutic vaccination of humans.

### STATEMENTS OF INVENTION

According to an aspect of the invention there is provided a vaccine composition comprising a polypeptide wherein said polypeptide is isolated from a human bacterial pathogen and is:
i) an amino acid sequence selected from the group consisting of: SEQ ID NO: 1, 2, 5, 6, 9, 10, 13, 14, 18, 19, 22, 23, 26, 27, 30, 31, 34, 35, 38, 39, 42, 43, 46, 47, 50, 51, 55, 56, 58, 59, 61, 62, 64, 66 or 68;
ii) an amino acid sequence as defined in i) above and which is modified by addition, deletion or substitution of one or more amino acid residues and which retains or has enhanced haem binding activity and/or reduced haemolytic activity.

A modified polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 35% identity, more preferably at least 40% identity, even more preferably at least 45% identity, still more preferably at least 50%, 60%, 70%, 80%, 90% identity, and most preferably at least 95%, 96%, 97%, 98% or 99% identity with the full length amino acid sequences illustrated herein.

In a preferred embodiment of the invention said antigenic polypeptide comprises or consists of an amino acid sequence as represented in SEQ ID NO: 1, 2, 5, 6, 9, 10, 13, 14, 18, 19, 22, 23, 26, 27, 30, 31, 34, 35, 38, 39, 42, 43, 46, 47, 50, 51, 55, 56, 58, 59, 61, 62, 64, 66 or 68.

In a preferred embodiment of the invention said antigenic polypeptide comprises or consists of an amino acid sequence as represented in SEQ ID NO: 1 or 2.

According to a further aspect of the invention there is provided a vaccine composition comprising a nucleic acid molecule comprising a nucleotide sequence that encodes an antigenic polypeptide isolated from a human bacterial pathogen wherein the nucleic acid molecule:
i) comprises a nucleotide sequence selected from the group consisting of: SEQ ID NO: 3, 4, 7, 8, 11, 12, 15, 16, 17, 20, 21, 24, 25, 28, 29, 32, 33, 36, 37, 40, 41, 44, 45, 48, 49, 52, 53, 54, 57, 60, 63, 65 or 67;
ii) comprises a nucleotide sequence wherein said sequence is degenerate as a result of the genetic code to the nucleotide sequence defined in (i); and
iii) is a nucleic acid molecule the complementary strand of which hybridizes under stringent hybridization conditions to the nucleotide sequence in i) and ii) above wherein said nucleic acid molecule encodes a haem binding protein.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecule comprises or consists of a nucleotide sequence as represented in SEQ ID NO: 3 or 4.

In a preferred embodiment of the invention said nucleic acid molecule comprises a transcription cassette comprising: a nucleic acid molecule that encodes said antigenic polypeptide operably linked to a promoter adapted for transcription of the nucleic acid molecule associated therewith.

In a preferred embodiment of the invention said promoter is a constitutive promoter.

In an alternative preferred embodiment of the invention said promoter is a regulatable promoter; preferably an inducible promoter and/or a tissue/cell specific promoter.

“Promoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors which have been shown to bind specifically to enhancer elements. The binding/activity of transcription factors (please see Eukaryotic Transcription Factors, by David S Latchman, Academic Press Ltd, San Diego) is responsive to a number of physiological/environmental cues. Promoter elements also include so called TATA box and RNA polymerase initiation selection sequences which function to select a site of transcription initiation. These sequences also bind polypeptides which function, inter alia, to facilitate transcription initiation selection by RNA polymerase.

In a preferred embodiment of the invention said promoter is a skeletal muscle specific promoter.

Muscle specific promoters are known in the art. For example, WO0009689 discloses a striated muscle preferentially expressed gene and cognate promoter, the SPEG gene. EP1072680 discloses the regulatory region of the myostatin gene. The gene shows a predominantly muscle specific pattern of gene expression. U.S. Pat. No. 5,795,872 discloses the use of the creatine kinase promoter to achieve high levels of expression of foreign proteins in muscle tissue. The muscle specific gene Myo D also shows a pattern of expression restricted to myoblasts. Further examples are disclosed in WO03/074711.

Preferably said consititutive promoter is selected from the group consisting of: Cytomegalovirus (CMV) promoter, α-globin RSV enhancer/promoter phosphoglycerate kinase (mouse PGK) promoter, alpha-actin promoter, SV40 promoter EF-1α promoter, ubiquitin promoter, transcription factor A (Tfam) promoter.

In a preferred embodiment of the invention said nucleic acid molecule is part of a vector.

In a preferred embodiment of the invention said vector is an expression vector adapted for expression of said nucleic acid molecule encoding said antigenic polypeptide according to the invention; preferably said nucleic acid molecule is operably linked to at least one promoter sequence.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, preferably selected from retro viridae baculoviridiae, parvoviridiae, picornoviridiae, herpesveridiae, poxyiridae, adenoviridiae, or picomnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent. Preferred vectors are derived from retroviral genomes [e.g. lentivirus] or are adenoviral based.

Viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al.; U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al. (1997) J. Virol 71(9):7128-7131; Lee, et al. (1997) Mol. Cell. Biol. 17(9):5097-5105; and Dreher, et al. (1997) J. Biol. Chem. 272(46); 29364-29371.

In a preferred embodiment of the invention said polypeptide or said nucleic acid molecule is isolated from a Gram negative human bacterial pathogen.

In a preferred embodiment of the invention said human bacterial pathogen is selected from the genus group consisting of: Neisseria, Moraxella, Escherichia, Salmonella, Shigella, Pseudomonas, Helicobacter, Legionella, Haemophilus, Klebsiella, Enterobacter, Cronobacter and Serratia.

In a preferred embodiment of the invention said human bacterial pathogen is Neisseria meningitidis.

In a preferred embodiment of the invention said human bacterial pathogen is Neisseria gonorrhoeae.

In a preferred embodiment of the invention said composition further comprises an adjuvant or carrier.

Adjuvants (immune potentiators or immunomodulators) have been used for decades to improve the immune response to vaccine antigens. The incorporation of adjuvants into vaccine formulations is aimed at enhancing, accelerating and prolonging the specific immune response to vaccine antigens. Advantages of adjuvants include the enhancement of the immunogenicity of weaker antigens, the reduction of the antigen amount needed for a successful immunisation, the reduction of the frequency of booster immunisations needed and an improved immune response in elderly and immunocompromised vaccinees. Selectively, adjuvants can also be employed to optimise a desired immune response, e.g. with respect to immunoglobulin classes and induction of cytotoxic or helper T lymphocyte responses. In addition, certain adjuvants can be used to promote antibody responses at mucosal surfaces. Aluminium hydroxide and aluminium or calcium phosphate has been used routinely in human vaccines. More recently, antigens incorporated into IRIV's (immunostimulating reconstituted influenza virosomes) and vaccines containing the emulsion-based adjuvant MF59 have been licensed in countries. Adjuvants can be classified according to their source, mechanism of action and physical or chemical properties. The most commonly described adjuvant classes are gel-type, microbial, oil-emulsion and emulsifier-based, particulate, synthetic and cytokines. More than one adjuvant may be present in the final vaccine product. They may be combined together with a single antigen or all antigens present in the vaccine, or each adjuvant may be combined with one particular antigen. The origin and nature of the adjuvants currently being used or developed is highly diverse. For example, aluminium based adjuvants consist of simple inorganic compounds, PLG is a polymeric carbohydrate, virosomes can be derived from disparate viral particles, MDP is derived from bacterial cell walls; saponins are of plant origin, squalene is derived from shark liver and recombinant endogenous immunomodulators are derived from recombinant bacterial, yeast or mammalian cells. There are several adjuvants licensed for veterinary vaccines, such as mineral oil emulsions that are too reactive for human use. Similarly, complete Freund's adjuvant, although being one of the most powerful adjuvants known, is not suitable for human use.

A carrier is an immunogenic molecule which, when bound to a second molecule augments immune responses to the latter. The term carrier is construed in the following manner. A carrier is an immunogenic molecule which, when bound to a second molecule augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen.

In a preferred embodiment of the invention said adjuvant is selected from the group consisting of aluminium hydroxide, aluminium or calcium phosphate.

In a preferred embodiment of the invention said adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative embodiment of the invention said adjuvant is a TLR agonist such as CpG oligonucleotides, flagellin, monophosphoryl lipid A, poly I:C and derivatives thereof.

In a preferred embodiment of the invention said adjuvant is a bacterial cell wall derivative such as muramyl dipeptide (MDP) and/or trehalose dicorynomycolate (TDM).

In a preferred embodiment of the invention said antigenic polypeptide comprises or consists of the amino acid sequence selected from the group consisting of SEQ ID NO: 1, 2, 5, 6, 9, 10, 13, 14, 18, 19, 22, 23, 26, 27, 30, 31, 34, 35, 38, 39, 42, 43, 46, 47, 50, 51, 55, 56, 58, 59, 61, 62, 64, 66 or 68 for use in the production of opsonins.

In a preferred embodiment of the invention said antigenic polypeptide comprises or consists of SEQ ID NO: 1 or 2.

In an alternative preferred embodiment of the invention said antigenic polypeptide is encoded by a nucleic acid molecule comprising or consisting of the nucleotide sequence selected from the group consisting of SEQ ID NO: 3, 4, 7, 8, 11, 12, 15, 16, 17, 20, 21, 24, 25, 28, 29, 32, 33, 36, 37, 40, 41, 44, 45, 48, 49, 52, 53, 54, 57, 60, 63, 65 or 67.

Preferably said nucleic acid molecule comprises SEQ ID NO: 3 or 4.

In a preferred embodiment of the invention said opsonin is an antibody.

According to an aspect of the invention there is provided a method for immunizing a human against a pathogenic bacterial species comprising:
i) administering an effective amount of a dose of a vaccine composition according to the invention to a human subject to induce protective immunity; optionally
ii) administering one or more further dosages of vaccine to said subject sufficient to induce protective immunity.

According to a further aspect of the invention there is provided a vaccine composition according to the invention for use in the treatment of Gram negative bacterial pathogenic infection in a human subject.

According to a further aspect of the invention there is provided a method for the production of an opsonin to an antigen isolated from a human bacterial pathogen comprising:
i) providing a vaccine composition according to the invention;
ii) administering an effective amount of said composition to a human subject sufficient to induce opsonin production.

The vaccine compositions of the invention can be administered by any conventional route, including injection, intranasal spray by inhalation of for example an aerosol or nasal drops. The administration may be, for example, intravenous, intraperitoneal, intramuscular, intracavity, subcutaneous, or intradermally. The vaccine compositions of the invention are administered in effective amounts. An “effective amount” is that amount of a vaccine composition that alone or together with further doses, produces the desired response. In the case of treating a particular bacterial disease the desired response is providing protection when challenged by an infective agent.

The amounts of vaccine will depend, of course, on the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used sufficient to provoke immunity; that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of vaccine administered to a subject can be chosen in accordance with different parameters, in particular in accordance with the mode of administration used and the state of the subject. In the event that a response in a subject is insufficient at the initial doses applied, higher doses (or effectively higher doses by a different, more localized delivery route) may be employed to the extent that patient tolerance permits.

In general, doses of vaccine are formulated and administered in effective immunizing doses according to any standard procedure in the art. Other protocols for the administration of the vaccine compositions will be known to one of ordinary skill in the art, in which the dose amount, schedule of injections, sites of injections, mode of administration and the like vary from the foregoing. Administration of the vaccine compositions to mammals other than humans, (e.g. for testing purposes or veterinary therapeutic purposes), is carried out under substantially the same conditions as described above.

In a preferred embodiment of the invention there is provided a vaccine composition according to the invention that includes at least one additional anti-bacterial agent.

In a preferred embodiment of the invention said agent is a second different vaccine and/or immunogenic agent (for example a bacterial polypeptide and/or polysaccharide antigen).

According to a further aspect of the invention there is provided a polypeptide as herein described for use in the treatment of human bacterial infections or conditions that result from human bacterial infections.

In a preferred method of the invention said bacterial infection is caused by one or more Gram negative bacteria.

In a preferred method of the invention said Gram negative bacteria is selected from the genus group consisting of: Neisseria, Moraxella, Escherichia, Salmonella, Shigella, Pseudomonas, Helicobacter, Legionella, Haemophilus, Klebsiella, Enterobacter, Cronobacter, Serratia, Kingella and Pasturella.

Other species include Pseudomonas aeruginosa and other Pseudomonas species, Stenotrophomonas maltophila, Burkholderia cepacia and other Burkholderia species, Alcatigenes xylosoxidans, species of Acinetobacter, Enterobacteriaceae, Haemophilus, Moraxella, Bacteroids, Fransicella, Shigella, Proteus, Vibrio, Salmonella, Bordetella, Helicobactor, Legionella, Citrobactor, Serratia, Campylobacter, Yersinia and Neisseria. In another embodiment of the invention gram-negative bacteria include Enterobacteriaceae which is selected from the group consisting of organisms such as Serratia, Proteus, Klebsiella, Enterobacter, Citrobacter, Cronobacter, Salmonella, Providencia, Morganella, Cedecea and Escherichia coli.

In a preferred embodiment of the invention said human bacterial pathogen is Neisseria meningitidis.

In a preferred embodiment of the invention said human bacterial pathogen is Neisseria gonorrhoeae.

According to a further aspect of the invention there is provided a method to immunize a human subject comprising vaccinating said subject with an effective amount of the vaccine composition according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

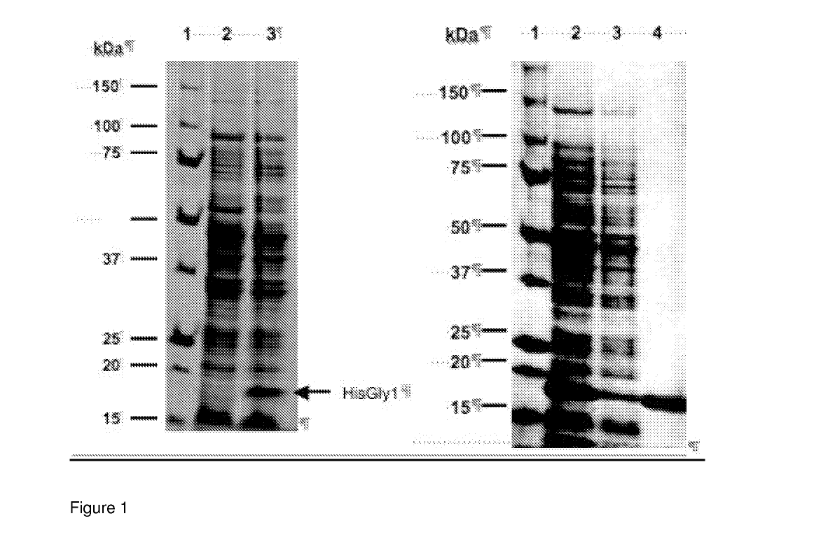

FIG. 1 illustrates Coomassie stained SDS-PAGE analysis of neisserial Gly1 protein production. Left panel shows Lane 1: Molecular weight marker, Lane 2: Uninduced concentrated culture supernatant; Lane 3: Induced concentrated culture supernatant. Right panel shows; Lane 1, molecular weight marker; Lane 2: supernatant applied to nickel-chelate column; Lane 3: Chelate column flow-through; Lane 4: Pooled chelate fractions eluted by imidazole gradient;

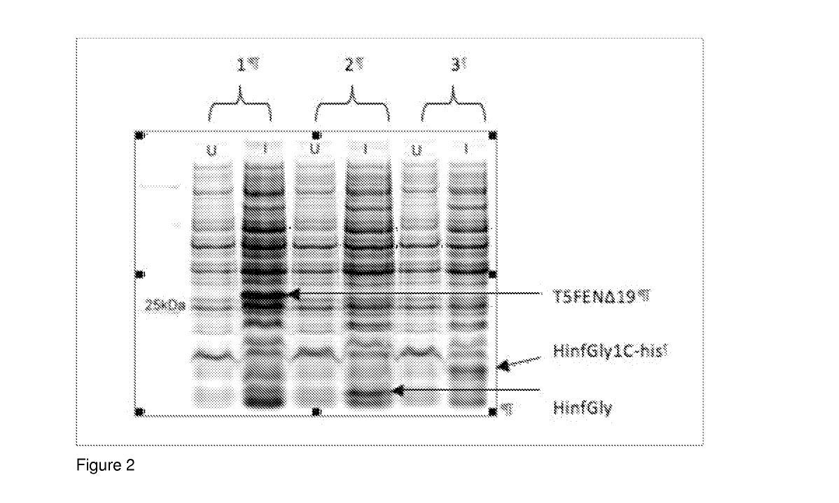

FIG. 2 illustrates expression test of the recombinant H. Influenzae Gly1ORF1 homologs analyzed by Coomassie stained SDS-PAGE. U-uninduced and I-induced cell pellet. 1—positive control—M72(λ) E. coli with T5FENΔ19 protein in pJONEX4 (a positive control for induction), 2—M72(λ) E. coli with the H. Influenzae Gly1ORF1 homolog, 3—M72(λ) E. coli with the H. influenzae Gly1ORF1 homolog with C-terminal histidine tag;

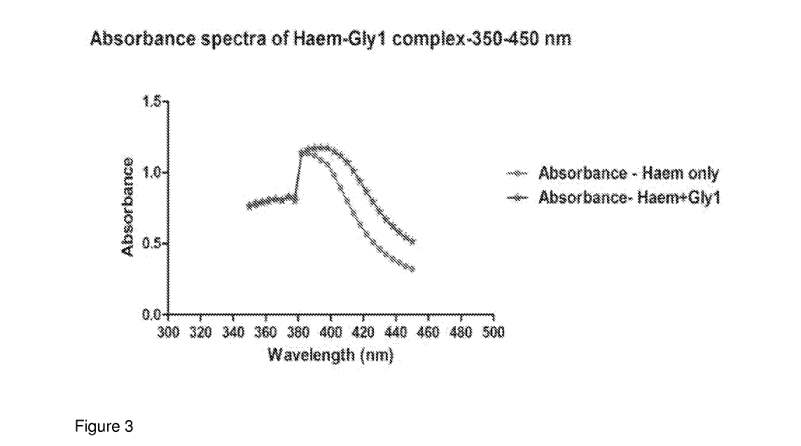

FIG. 3 illustrates spectra of hemin [haemin, circles] and the complex with N. meningitidis gly 1 protein [stars];

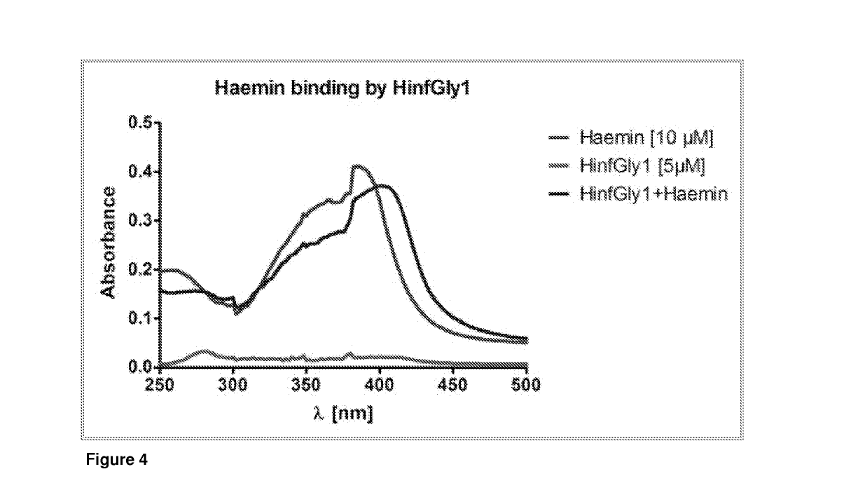

FIG. 4 illustrates spectra of hemin [haemin, circles] and the complex with H. influenzae gly 1 protein [stars]. Spectra of H. influenzae gly 1 protein alone [green] hemin [haemin] (red) and the complex (blue);

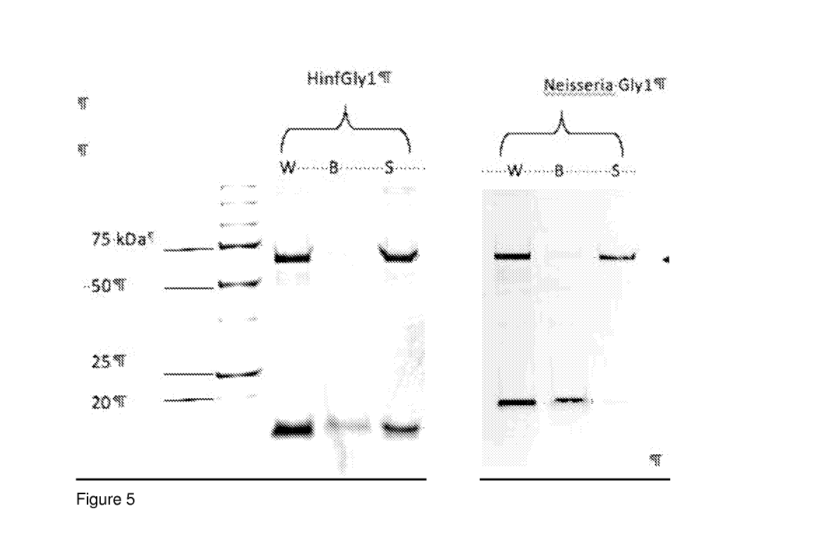

FIG. 5 illustrates Coomassie stained SDS-PAGE-analysis of hemin-agarose bead pull-down assay showing selective binding of H. influenzae (HinfGly1) and Neisseria meningitidis Gly1 to hemin beads, W—Gly1 (lower band) was mixed with BSA (upper band) before incubation with hemin beads. B—after incubation in Gly1/BSA solution, hemin beads were washed with PBS and boiled in SDS to release bound proteins. S—supernatant after pelleting the beads after incubation with BSA and Gly1 proteins;

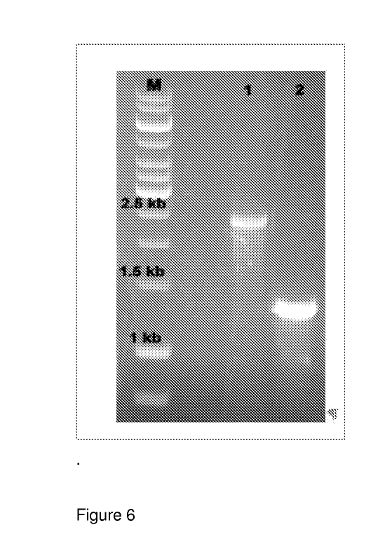

FIG. 6 illustrates authentication of Gly1 mutation in MC58 by PCR—Lane M—1 kb DNA ladder (Fermentas) with the molecular weight of the relevant bands labelled; Lane 1—PCR product of MC58ΔGly1 amplified with primers KOF and KOR; Lane 2—PCR product of MC58 wild type amplified with primers KOF and KOR;

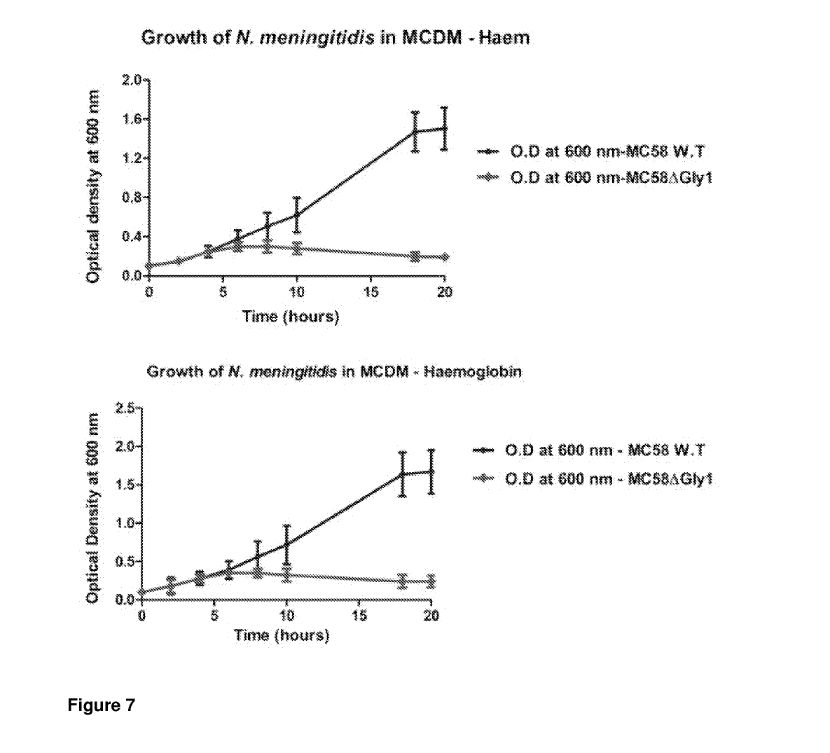

FIG. 7 illustrates growth of MC58 wild type (W.T.) and MC58ΔGly1 in MCDM—Upper Panel shows the growth of MC58 W.T. (blue curve) and MC58ΔGly1 (red curve) in media containing haem as the only source of iron over a 20 hour period is shown. Lower panel shows the growth of MC58 W.T. (blue curve) and MC58ΔGly1 (red curve) in media containing haemoglobin as the only source of iron over a 20 hour period is shown;

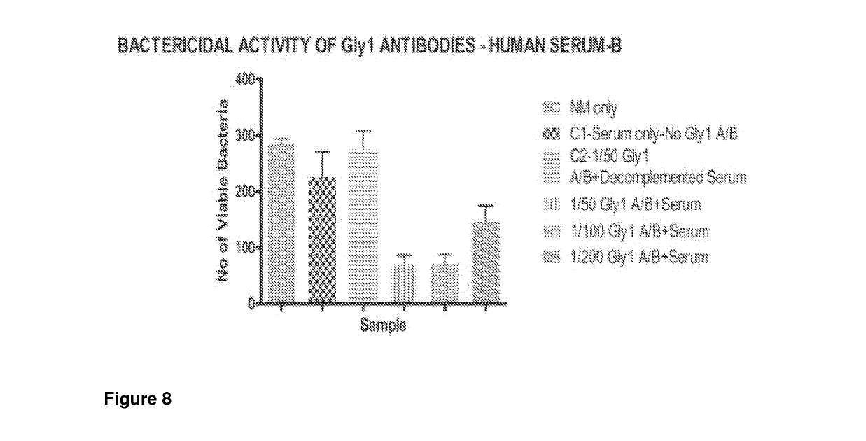

FIG. 8 illustrates Bactericidal activity of Gly1 antibodies—Bacteria plated directly (NM—N. meningitidis only; untreated control group bacteria), or treated with further controls C1; serum without anti-gly1 antibodies, C2; 1/50 dilution of Gly1 antibodies with decomplemented serum). However, the presence of antigly1 antibodies and serum leads to a bacteriocidal action. A reduction in the number of viable bacteria in samples containing the antibodies and the complement is seen at various dilutions of anti-Gly1 antibodies;

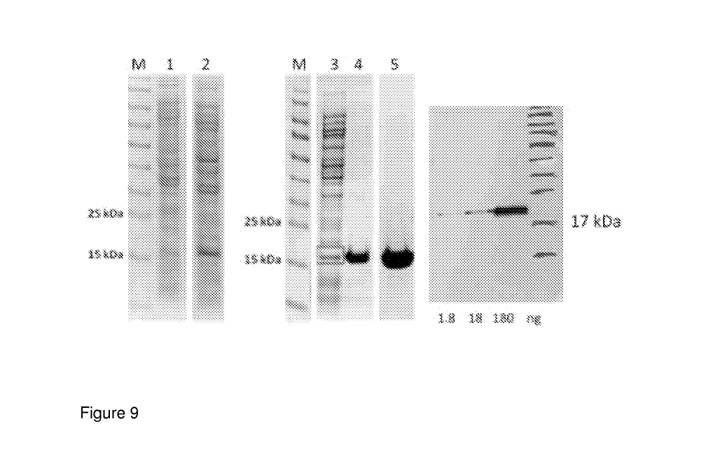

FIG. 9 illustrates Coomassie stained SDS-PAGE-analysis of expression and purification of a Salmonella enterica Gly1 homologue in E. coli. M indicates molecular weight markers, Lane 1-uninduced and Lane 2 induced, induced cell pellets of M72(λ) E. coli with the S. enterica Gly1 gene cloned into pJONEX-CHIS so as to include a C-terminal histidine tag. Lane 3 shows total soluble proteins applied to a nickel chelate column. Lanes 4 and 5 show Salmonella Gly1-homologue after purification on nickel chelate and anion exchange columns respectively.

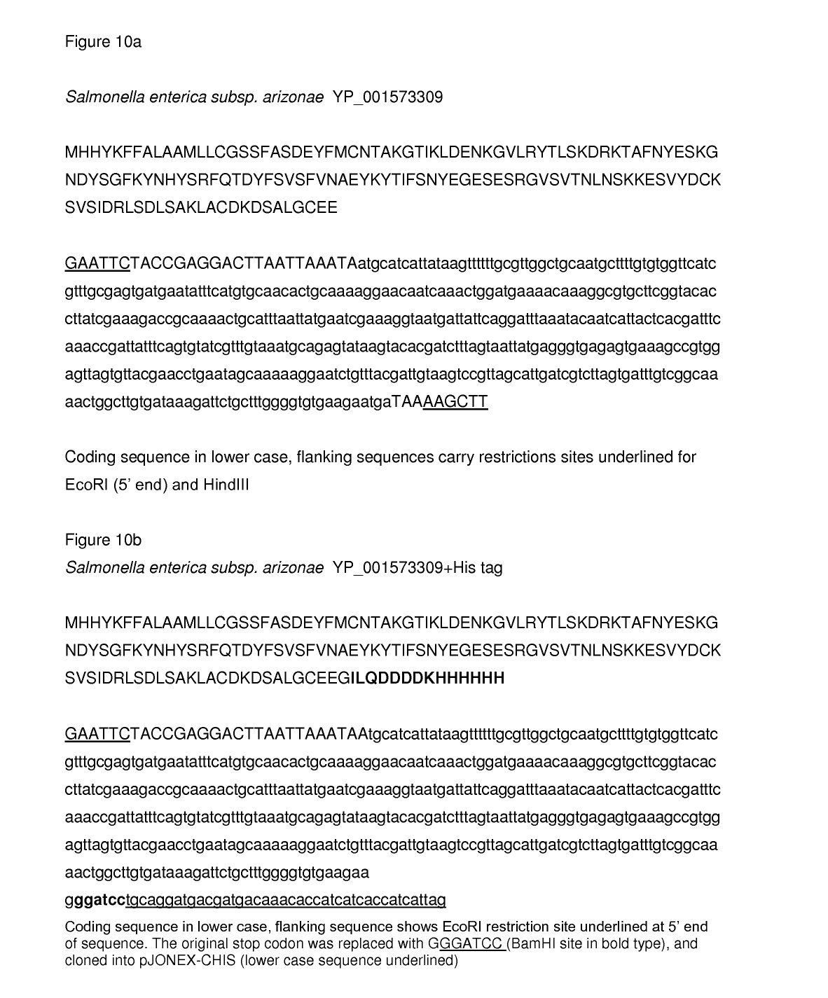

FIG. 10 illustrates nucleotide sequences and the cloning of Salmonella entrica Gly1 homologue.

### MATERIALS AND METHODS

Cloning:

The plasmid pJONEX4 (J. R. Sayers, F. Eckstein, Nucleic Acids Res. 19, 4127 (1991) was digested with BamHI and HindIII and ligated with a duplex DNA consisting of two oligonucleotides

(C1 5′ GATCCTGCAGGATGACGATGACAAACACCATCATCACCATCATTAG and C2 5′ AGCTCTAATGATGGTGATGATGGTGTTTGTCATCGTCATCCTGCAG)

to create a plasmid designated pJONEX-CHIS which was transfected into competent E. coli cells and the progeny plasmids were characterized by DNA sequencing. PCR primers F1 (TTTCGAATTCTAAGGAAATACGATGAA) and R1 (ATCCGCCCCGGGATCCCGGAAAAAT), were used to PCR-amplify the gly1orf1 gene from N. meningitidis MC58 using standard methods, and the resulting fragment digested with EcoRI and BamHI (sites underlined) and ligated into the EcoRI and BamHI of pJONEX-CHIS. This results in an in-frame fusion of the Gly1-coding region with a C-terminal enterokinase recognition site, six histidine residues, and stop codon. The newly constructed DNA was transfected into M72(λ) at 28° C. on LB-ampicillin plates. The resulting recombinant construct was designated pJONGLY1 his was sequenced (Core Genomics Facility, University of Sheffield Medical School) using standard M13 forward and reverse primers.

Haemphilus influenzae (Genbank accession no. AJ627386) strain was kindly donated by Dr David Wyllie from The University of Oxford. The cultures were streaked out on the Chocolate Agar Plates and incubated overnight at 37° C. in increased CO₂ conditions. A single colony was transferred with a sterile loop into 50 μL of TE buffer (10 mM Tris-HCl, pH 8/1 mM EDTA) with addition of 0.1% (w/v) SDS and cell suspension was lysed by incubation for 10 min at 95° C. This lysate was used as a template for PCR. The wild-type H. influenzae gly1 (HinfGly1 WT) was obtained using the primers Gly1_hinf_K.f (AATAGGTACCTGAGGAGAAACAAATGACAAAATTACTCACTCACTATTGGAGC)+Hinf_gly1.r (TATTCGATCCCTCAAGCTTTCATACTGCGACGT). The C-terminal his-tagged H. influenzae gly1 (HinfGly1 C6H-tag) was obtained using the primers Gly1_hinf_K.f and Hinf_Gly1H6.r #(AAATGTCGACGCTTATTTTATCTCTATTTACGCTTATGA) using standard protocols. The product HinfGly1 WT was digested with KpnI and HindIII and ligated into pJONEX4 (cut with the same restriction enzymes) to generate pJONHGLY, while HinfGly1 C6H-tag was digested with KpnI+SalI and ligated into pJONEX4-CHIS generating pJONHGLY-his).

Protein Production:

An overnight culture (100 mL) of M72(pJONGLY1his) in 5YT media containing 100 □g ml⁻¹ carbenicillin was inoculated into a 2 L fermenter containing 1.5 L of 5YT/carbenicillin media, incubated at 30° C., stirred at 750 rpm and provided with an air supply of 2 L min⁻¹. At mid-log phase the temperature was increased to 42° C. for 3 hr. The cells were then removed from the spent supernatant by centrifugation at 10,000×g for 20 min at room temperature. A similar approach was used for the other gly1 homologues.

Protein Purification:

C-terminal his-tagged proteins were purified from the clarified supernatant using metal affinity chromatography essentially as described (D. N. Arvidson, R. F. Pearson, C. G. Arvidson, Acta Crystallogr. D Biol. Crystallogr. 59, 747 (2003)). Untagged proteins were purified from the supernatant using ion exchange chromatography on cation and anion exchange columns using standard protocols, purification was monitored by SDS PAGE. Alternatively, proteins were extracted from the cell pellet by resuspending in packed cells in 50 MM Tris HCl, pH 8, 200 mM NaCl (10 ml per g of packed cells) then solid guanidinium hydrochloride with stirring until the suspension cleared. The viscosity was reduced by sonication and debris removed by centrifugation at 43,000×g for 30 min at room temperature. The supernatant was then removed, diluted to approx. 3 M in guanidinium hydrochloride, centrifuged as before and applied to a nickel chelate affinity chromatography column. The guanidinium hydrochloride was removed by washing the column in 10 volumes of compatible buffer, followed by washing with either an acid or imidazole gradient to effect elution.

Haem (Hemin) Binding Assays:

The change in the UV-visible spectrum of free and gly1 bound hemin was examined by spectroscopy and using hemin-agarose beads as an affinity matrix to pull down Gly1 proteins from a mixture of GLy1 with carrier BSA.

Production of Antibodies:

Protein samples were allowed to thaw on ice and buffer exchanged for phosphate buffered saline. The protein was than used to immunize rabbits (BioSery UK Ltd, Sheffield).

Gene Knockouts:

Meningococci (strain MC58) were routinely grown on blood and chocolate agar plates (Oxoid, UK) or GC agar plates supplemented with 1% (w/v) Vitox (Oxoid, UK). The liquid media typically used for growing meningococci were brain heart infusion media (Oxoid, UK) or Meningococcal chemically defined media (MCDM) with iron supplements. A 1.3 kb fragment containing gly1 gene and 500 bp fragments upstream and downstream of the Gly1 open reading frame was amplified from the genomic DNA of MC58 strain of N. meningitidis using the following primers; Gly1KOF-5′-GCCGACGGCAAAACGGTTCA-3′ and Gly1KOR-5′-CCAAACCGGCAGGCGCAAA-3′ by PCR. The amplified fragment was cloned blunt-ended on to Ssp1-digested plasmid pKNT25 (Karimova, G, Dautin, N & Ladant, D. J. Bacteriol. 2005 April; 187(7): 2233-2243) yielding pKNT25Gly1. pKNT25Gly1 was further digested with Ssp1 which cuts in the middle of the gly1 gene to get the linear plasmid pKGly1ΔSsp1. The erythromycin resistance cassette (ermR) was amplified from plasmid pFNR1 using the primers Ery_For—5′-GGGGCCCTGTGTTGATAGTGCAGTATCTTAAAA-3′ and Ery_Rev—5′-GGGGCCCTATATCTAATAATTTATCTCCATTCCCTT-3′ by PCR. The amplified fragment ermR was ligated into the plasmid pKGly1 Ssp1 site producing plasmid pKGly1ermR. The 2.5 kb fragment gly1-ermR containing ermR flanked by fragments of gly1 gene was amplified using the primers Gly1 KOF-5′-GCCGACGGCAAAACGGTTCA-3′ with Gly1KOR-5′-CCAAACCGGCAGGCGCAAA-3′ by PCR.

N. meningitidis MC58 grown overnight in chocolate agar plates were resuspended in proteose-peptone broth (Proteose peptone No 3 (BD Biosciences, NJ, USA)—15 mg ml⁻¹; Di-potassium hydrogen phosphate—4 mg ml⁻¹; Potassium di-hydrogen orthophosphate—1 mg ml⁻¹; NaCl—5 mg ml⁻¹; 1% (w/v) Vitox (Oxoid, UK) and 10 mM MgCl₂) to a final OD 600ₙₘ of 0.2. To 250 μl of the suspension 1 μg of the amplified gly1-ermR fragment was added. The bacterial suspension with DNA was then incubated at 37° C. and 5% CO₂ for 4 hours. A control transformation with just TE buffer without DNA was also performed simultaneously. After the incubation, 100 μl of both the suspensions were plated on to GC agar plates supplemented 1% Vitox (Oxoid, UK) containing 5 μg ml⁻¹ of erythromycin (Sigma, Mo., USA). The plates were incubated overnight at 37° C. and 5% CO₂. The colonies obtained were re-streaked onto fresh GC agar plates containing erythromycin to discount the possibility of the presence of wild type alleles. After 3 rounds of selection the colonies obtained were combined to eliminate the possibility of phase variation and stored at −80° C.

The mutagenesis was authenticated by amplifying the genomic DNA of MC58ΔGly1 with primers Gly1KOF and Gly1KOR and analyzing the increase in size of the amplified fragment due to the insertion of emrR cassette into the middle of gly1 gene. The presence of emrR cassette was confirmed by amplifying the fragment with primers Ery_for and Ery_rev. The insertional mutagenesis was further confirmed by sequencing the amplified fragments with primers Gly1KOF, Gly1KOF, Ery_for and Ery_rev.

Growth of N. Meningitidis MC58 W.T and MC58ΔGly1 in MCDM

SOLUTION A contained 166 mM K₂HPO₄ and 62 mM KH₂PO₄ dissolved in 475 ml of distilled water. The solution was sterilized by autoclaving. SOLUTION B: contained 1.6 mM MgSO₄ 116 mM NH₄Cl and 56 μM MnSO₄ dissolved in 475 ml of distilled water. The solution was sterilized by autoclaving. SOLUTION C: Contained 350 μM FeSO₄/haem/haemoglobin 250 μM CaCl₂ and 7.5 mM Na₂S₂O₃ dissolved in 20 ml of distilled water. The solution was then filter sterilized. SOLUTION D (supplements): contained 1.4 mM arginine, 0.06 mM cysteine, 1 mM glutamate, and 1 mM glycine dissolved in 20 ml of distilled water. The solution was filter sterilized. SOLUTION E (vitamins): Contained thiamine hydrochloride—0.02 g, calcium pantothenate—0.019 g, and thiamine pyrophosphate chloride (cocarboxylase)—0.0048 g dissolved in 100 ml of distilled water. The solution was then filter sterilized SOLUTION F. This solution contained; biotin—0.015 g dissolved in 50 ml of ethanol and the final volume was made up to 100 ml with sterile distilled water. The solution was filter sterilized.

MCDM was prepared by mixing 228 ml of solution A, 228 ml of solution B, 20 ml of solution C, 8.3 ml of solution D, 3.3 ml of solution E and 8.3 ml of solution F. Glucose and NaHCO₃ were added to a final concentration of 30 mM. The pH of the solution was adjusted to 7.2 with HCl. Make up the volume to 500 ml with sterile distilled water.

Growth of N. Meningitidis in Meningococcal Chemically Defined Media (MCDM)

MC58 wild type and MC58ΔGly1 were routinely grown in GC Agar plates supplemented with 1% Vitox (Oxoid, UK) with or without 5 μg/ml Erythromycin (Sigma, Mo., USA—for the mutant). The bacteria were grown overnight at 37° C. and 5% CO₂. The bacteria thus grown were harvested in Meningococcal Chemically Defined Media without any external iron source (MCDM-0) and were further diluted in pre-warmed MCDM containing the respective sources of Iron. The cultures were grown at 37° C. and the growth was calculated by measuring the absorbance at 600 nm. The absorbance was measured at 2, 4, 6, 8, 10, 18 and 20 hours after inoculation. Either hemin or haemoglobin were added as iron sources.

Serum Bacteriocidal Antibody Assay

N. meningitidis MC58 was grown overnight on chocolate agar plates or GC agar plates supplemented with 1% vitox (Oxoid, UK). The bacteria were harvested and diluted in PBS containing 1% fetal calf serum (FCS). Approximately 1500 c.f.u of meningococci were incubated in PBS containing 1% FCS in the presence of different dilutions of Gly1 antibodies (1/10, 1/50, 1/100, 1/200) and human serum which acts as the source of complement in 96 well plates. The final reaction volume was made up to 100 μl with PBS. The controls without antibodies and or the serum were always included. After an hour of incubation at 37° C. and 5% CO₂ a 20 μl sample from each reaction was plated onto blood agar plates and were grown for 16 hours at 37° C. and 5% CO₂. Following the incubation, the viable cells were enumerated and graphs were plotted. The experiment was done in triplicates at three different time points and the mean and standard errors were plotted.

Construction and Synthesis of a Salmonella entrica Gly 1 Homologue

A synthetic DNA encoding the Salmonella enterica subsp. arizonae serovar amino acid sequence (YP_{—}001573309), a Neisseria meningitidis GLY1ORF1 homologue, was constructed by gene synthesis (Eurofins MWG) and cloned into the pJONEX4 plasmid via flanking EcoRI and HindIII sites. Similarly, a DNA construct in which the stop codon was replaced with a BamHI site so as to allow expression of YP_{—}001573309 as a fusion protein sequence in frame fusion with a C-terminal six histidine tag was prepared and subcloned into pJONEX-CHIS. The recombinant plasmids were used to produce tagged and untagged S. eneterica Gly1 homologue (SalGLY1).

### EXAMPLES

Recombinant Gly1 proteins were readily produced in E. coli as shown in FIGS. 1 and 2. The Gly1 proteins from N. meningitidis and H. influenzae caused as shift in the visible spectrum of hemin as demonstrated in FIGS. 3, 4 indicating that they may bind it as a ligand. This was confirmed using pull-down assays with hemin-agarose beads as shown in FIG. 5.

A deletion of the gly1 gene was made in N. meningitidis (FIG. 6). This mutant, unlike the wild type parent, was unable to grow on either hemin or haemoglobin as an iron source (FIG. 7).

Polyclonal antibodies raised against recombinant N. meningitidis Gly1 protein in rabbits were able to mediate serum bacteriocidal activity as shown in FIG. 8.

FIG. 9 illustrates a SDS-PAGE gel showing protein size markers (M), total protein from M72 cells carrying recombinant gene for C-his tagged SalGly1 before (lane 1) and after (lane 2) induction. After lysis, soluble protein (Lane 3), protein was purified by nickel chelate (Lane 4) and ion exchange chromatography (Lane 5). The right hand panel shows the results of a western blot using the indicated amounts of SalGly1 protein with primary antisera raised in rats at a dilution of 1:5000.

1. A vaccine composition comprising an antigenic polypeptide wherein said antigenic polypeptide is isolated from a human bacterial pathogen and comprises:
i) an amino acid sequence selected from the group consisting of: SEQ ID NO: 1, 2, 5, 6, 9, 10, 13, 14, 18, 19, 22, 23, 26, 27, 30, 31, 34, 35, 38, 39, 42, 43, 46, 47, 50, 51, 55, 56, 58, 59, 61, 62, 64, 66, and 68;
ii) an amino acid sequence as defined in i) above and which is modified by addition, deletion or substitution of one or more amino acid residues and which retains or has enhanced haem binding activity and/or reduced haemolytic activity.

2. The vaccine composition according to claim 1, wherein said antigenic polypeptide comprises or consists of an amino acid sequence as represented in SEQ ID NO: 1, 2, 5, 6, 9, 10, 13, 14, 18, 19, 22, 23, 26, 27, 30, 31, 34, 35, 38, 39, 42, 43, 46, 47, 50, 51, 55, 56, 58, 59, 61, 62, 64, 66 or 68.

3. (canceled)

4. The vaccine composition according to claim 1, wherein the vaccine composition comprises a nucleic acid molecule encoding the antigenic polypeptide, wherein the nucleic acid molecule:
i) comprises or consists of a nucleotide sequence shown in SEQ ID NO: 3, 4, 7, 8, 11, 12, 15, 16, 17, 20, 21, 24, 25, 28, 29, 32, 33, 36, 37, 40, 41, 44, 45, 48, 49, 52, 53, 54, 57, 60, 63, 65 or 67;
ii) comprises a nucleotide sequence degenerate as a result of the genetic code to the nucleotide sequence defined in (i); or
iii) comprises a nucleic acid molecule complementary strand to the nucleotide sequence in i) or ii) and which hybridizes under stringent hybridization conditions to the nucleotide sequence in i) or ii) above wherein said nucleic acid molecule encodes a haem binding protein.

5. (canceled)

6. The vaccine composition according to claim 4, wherein said nucleic acid molecule comprises a transcription cassette comprising: the nucleic acid molecule that encodes said antigenic polypeptide operably linked to a promoter adapted for transcription of the nucleic acid molecule that encodes said antigenic polypeptide.

7. The vaccine composition according to claim 6, wherein said nucleic acid molecule is part of an expression vector.

8. The vaccine composition according to claim 4, wherein said antigenic polypeptide or nucleic acid molecule is isolated from a Gram negative human bacterial pathogen.

9. The vaccine composition according to claim 8 wherein said Gram negative human bacterial pathogen is selected from the genus group consisting of: Neisseria, Moraxella, Escherichia, Salmonella, Shigella, Pseudomonas, Helicobacter, Legionella, Haemophilus, Klebsiella, Enterobacter, Cronobacter, Serratia, Kingella and Pasturella.

10. The vaccine composition according to claim 9 wherein said human bacterial pathogen is Neisseria meningitides or Neisseria gonorrhoeae.

11. (canceled)

12. The vaccine composition according to claim 1, wherein said composition further comprises an adjuvant or carrier.

13. The vaccine composition according to claim 12, wherein said adjuvant comprises:
aluminium hydroxide,
aluminium,
calcium phosphate,
a cytokine selected from the group consisting of GMCSF, interferon gamma, interferon alpha, interferon beta, interleukin 12, interleukin 23, interleukin 17, interleukin 2, interleukin 1, TGF, TNFα, and TNFβ,
a TLR agonist, or
a bacterial cell wall derivative.

14.-21. (canceled)

22. A method for immunizing a human against a pathogenic bacterial species comprising:
i) administering an effective amount of a dose of the vaccine composition of claim 1 to a human subject to induce protective immunity; and optionally
ii) administering one or more further dosages of the vaccine composition to said subject sufficient to induce protective immunity.

23. (canceled)

24. A method for the production of an opsonin to an antigen derived from a human bacterial pathogen comprising:
i) providing the vaccine composition of claim 1; and
ii) administering an effective amount of said vaccine composition to a human subject sufficient to induce opsonin production.

25. The method according to claim 24, wherein said composition includes at least one additional anti-bacterial agent.

26. The method according to claim 25, wherein said agent is a second different vaccine and/or immunogenic agent.

27.-32. (canceled)

33. A method for treating a human bacterial infection or a condition resulting from the human bacterial infection, comprising:
administering an effective amount of the vaccine composition of claim 1 to a human sufficient to treat the human bacterial infection or a condition resulting from the human bacterial infection.

34. The method of claim 33, wherein the human bacterial infection is caused by one or more Gram negative bacteria.

35. The method of claim 34, wherein the Gram negative bacteria is selected from the genus group consisting of: Neisseria, Moraxella, Escherichia, Salmonella, Shigella, Pseudomonas, Helicobacter, Legionella, Haemophilus, Klebsiella, Enterobacter, and Serratia.
